(19) United States
(12) Patent Application Publication
(10) Pub. No.: US 2026/0112503 A1
(43) Pub. Date: Apr. 23, 2026

(54) METHOD TO IDENTIFY A CONDITION IN A HAIR SAMPLE

(71) Applicant: Matthew T. Nogle, Lebanon, OH (US)

(72) Inventor: Matthew T. Nogle, Lebanon, OH (US)

(21) Appl. No.: 19/364,755

(22) Filed: Oct. 21, 2025

Related U.S. Application Data
(60) Provisional application No. 63/710,070, filed on Oct. 22, 2024.

(51) Int. Cl. G16H 50/30; G06N 20/00; G06T 7/00; G16H 30/40

(52) U.S. Cl. CPC G16H 50/30; G06N 20/00; G06T 7/0012; G16H 30/40; G06T 2207/10004; G06T 2207/10056; G06T 2207/30024

(57) ABSTRACT

A method of detecting disease through analysis of hair images can indicate specific diseases is an intriguing topic intersecting dermatology, diagnostic medicine, and technology. By obtaining a hair from the body and analyzing images for fungus as an indicator of various health conditions. Disclosed herein is a methods relating to the classification of hair samples which can be used to identify and to diagnose conditions or to support treatment-related decisions.

### BACKGROUND OF THE INVENTION

Neurodegenerative and neuro musculature and autoimmune diseases such as Alzheimer and Parkinson's is one of many of the leading causes of mortality worldwide; yet for many patients, the process of simply clearing the first step of obtaining an early accurate diagnosis is often a frustrating and time-consuming experience. An inaccurate or late diagnosis of disease can lead to unnecessary follow-up procedures, including costly medical procedures, not to mention unnecessary emotional distress to the patient. In the case of certain diseases, there are hundreds of thousands of patient suffering from these diseases in the United States, many of which diagnosed earlier and may remain undiagnosed or misdiagnosed and mistreatment is prescribed without treating and missing the actual disease process.

Hair has long been a subject of interest in various scientific and medical fields. Traditionally, its primary role has been as a biological marker for age, gender, and genetic traits. However, recent advances in technology and diagnostic methods have prompted a renewed focus on the potential of hair analysis in diagnosing diseases. With this is mind, the instant invention is directed at improving the field of detection of disease through the hair and peri hair tissue to diagnose or indicate a proper and faster diagnosis.

### SUMMARY OF THE INVENTION

The instant invention process provides that a 2D picture, semi 3D video and digital live modeling of hair provides an indication to specific diseases correlating to hair characteristics, including texture, color, and structural cellular changes, identification of foreign pathogens such as fungi or bacteria, and whether organisms such as fungi, co-exist to infer underlying health conditions. This process hypothesis is derived by reviewing relevant research, examining methodologies, and discussing practical implications. Continued treatment costs and complications due to the need for lifelong drug therapy to improper treatment can cause further economic and physical harm. The current invention can sample in 3D without the need of cutting at the sample.

In some embodiments, the invention provides a method to identify fungal induced infections at the preclinical and subclinical stage of development. This stage is often overlooked by standard conventional testing. Also, ADHD symptoms, localized immune suppression with correlation to skin and gastro intestinal cancers and fungal induced drug amphetamine dependence and generalized neurological mental depression causuality can be understood and diagnosed on symptomatic and physical sign process. This will save valuable patient suffering and monetary expence in the healthcare system and rehabilitation system.

During prolonged drug use such as amphetamines, fungi or bacteria may propagate thus lowering systematic levels of natural neurochemicals and hormones by the fungi or bacteria digesting these neurochemicals and hormones. This deficiency contributes to the patient's desire for increased drug consumption. The patient is now consuming drugs for the propogation of fungi or bacteria colonies. This furthers the addition cascade.

On a localized tissue level, fungal or bacterial colonization on hair allows for the fungi or bacteria to hide from the immune system somewhat, especially those immuno compromised. The fungi or bacteria is then able to produce immune suppressive chemical that stops the body's natural defenses. This also likely interferes with the normal blood serum testing route for fungal or bacterial infection. The normal tests produce a false negative report. This localized tissue immunosuppression is also likely to produce an increased risk for skin and gastrointestinal and other cancers.

A primary method of fungal or bacterial hair infections casuality form the discovery of this invention is the dissemination of bacterial pathogens that are likely dissolved in saliva. The fungal or bacterial infection is noted on facial hairs with close proximity to the oral cavity, and close proximity to saliva.

The method is to test provide a subclinical and preclinical fungal or bacterial infection indication and justify the use of antifungal or antibacterial medications to lower the patient's desire for street and prescription drugs by raising the serum levels and central nervous system and peripheral levels of natural hormones and vitamins and neurotransmitters. Further, to test for pre and subclinical fungal or bacterial infections allows for higher efficiency in the use of antimicrobials, antifungals and antibiotics for more successful treatment outcomes. Finally, early detection and prevention and future vaccine production for pathogenic fungi or bacteria is an enabled goal of prevention the disease which can be accomplished through the early diagnosis.

### BRIEF DESCRIPTION OF THE DRAWINGS

FIG. 1 is a link to a standard microscopic view of human hair by traditional microscopy.

FIG. 2 shows a proprietary visualization of follicular portion of hair root. Label A indicates abnormal secondary hair shaft lateral growth. Label B indicates melanin fungal pigment produced by A. niger.

FIG. 3 shows a keratin cells of a human hair Label A indicates pathogenic bacteria Borrelia burgdorferi. Label B indicates A. niger.

FIG. 4. Proprietary medium magnification of pathogenic human hair. Label A indicates A. burgdorferi. Label B indicates A. niger.

FIG. 5. Proprietary high magnification of the initial formation of the fungal bacterial complex attached to the periphery of human hair. Label A indicates A. burgdorferi. Label B indicates A. niger melanin. Label C indicates the symbiotic complex created by the two species.

FIG. 6. Proprietary medium magnification view of human hair showing pathologic destruction of the keratin structure and invasion by A. niger. B A. niger.

FIG. 7. shows a proprietary medium magnification of extracted human hir follicular visualizing internal structures and peri-follicular tissues. A pathologic tissue destruction. B peri-follicular tissues.

FIG. 8. proprietary medium magnification of pathologic extracted hair. B A. niger tissue destruction C expanded medium high magnification to FIG. 9.

FIG. 9. High magnification of whole extracted human hair with A. niger tissue destruction. B A. niger associated tissue destruction.

shows a standard published pictures of pathologic structures associated with Alzheimer's disease.

FIG. 10 shows medium high magnification of extracted pathologic hair showing (C) circular structures forming forming producing fungi A. nigher and A. burgdorferi.

FIG. 11 shows medium high magnification of extracted pathologic hair showing (C) circular structures forming forming producing fungi A. nigher and A. burgdorferi.

FIG. 12 shows medium high magnification of extracted pathologic hair showing (C) circular structures forming produced by fungi A. nigher and bacterium A. burgdorferi.

FIG. 13 shows medium to high magnification of peri-thrix tissue that was attached to the hair follicle after the extraction labeled “MK” melanotic ketatotic circular tissue clumps noted. These are believed to be the mechanism of budding type reproduction spreading the organisms deep into tissues as possible the blood stream.

FIG. 14 shows medium to high magnification of peri-thrix tissue that was attached to the hair follicle after the extraction labeled “MK” melanotic ketatotic circular tissue clumps noted. These are believed to be the mechanism of budding type reproduction spreading the organisms deep into tissues as possible the blood stream.

FIG. 15 shows medium to high magnification of peri-thrix tissue that was attached to the hair follicle after the extraction labeled “MK” melanotic ketatotic circular tissue clumps noted. These are believed to be the mechanism of budding type reproduction spreading the organisms deep into tissues as possible the blood stream. medium to high magnification of peri-thrix tissue that was attached to the hair follicle after the extraction (MK) melanotic ketatotic circular tissue clumps noted. These are believed to be the mechanism of budding type reproduction spreading the organisms deep into tissues as possible the blood stream.

FIG. 16 shows medium to high magnification of peri-thrix tissue that was attached to the hair follicle after the extraction labeled “MK” melanotic ketatotic circular tissue clumps noted. These are believed to be the mechanism of budding type reproduction spreading the organisms deep into tissues as possible the blood stream.

FIG. 17. shows what was a keratinocyte of the hair structure that was singled out and is being transformed into a melanotic circular structure outside of the hair structure labeled “MK”.

FIG. 18. depicts the STAR OF DAVID™” THE PIVOTAL AH HA SLIDE CORRELATING DISEASE PICTURE this shows similar structure to Alzheimer brain lesions.

FIG. 19 is a view of neurodegenerative like pleomorphic structures.

FIG. 20 is link to a standard published picture of a Parkinson's disease lesion

FIG. 21 show a structure made by the fungal bacteria complex. The structures are labelled as P.

FIG. 22 shows a structure made by the fungal bacteria complex. The structures are labelled as P.

FIG. 23 shows structure made by the fungal bacteria complex. The structures are labelled as P.

FIG. 24 shows structures made by the fungal bacteria complex. The structures are labelled as P.

FIG. 25 show structures made by the fungal bacteria complex. The structures are labelled as P.

FIG. 26 is is a link to published standard pictures of neuro degenerative multiple sclerosis disease lesions.

FIG. 27 shows Perithrix material.

FIG. 28 show similar shaped structures as the lymphocyte infiltrate In peripheral neuro degenerative disorders.

FIG. 29 show similar shaped structures as the lymphocyte infiltrate In peripheral neuro degenerative disorders.

FIG. 30 shows similar shaped structures as the lymphocyte infiltrate In peripheral neuro degenerative disorders.

FIG. 31 is a link to STANDARD PICTURES OF MYO DEGENERATIVE OR MUSCULAR DEGENERATIVE DISEASES

FIG. 32. Microscopic picture using the invention to find similar structures to neuro muscular and muscular degenerative diseases.

FIG. 33 is an extracted pathogenic whole untreated hair follicle.

FIG. 34. shows a close up magnification of and extracted human hair showing a pleomorphic round structure of Lyme disease bacteria Borrelli burgdorferi in co-infection with Aspergillus niger.

FIG. 35 shows a higher magnifications of various aspects of an extracted pathogenic whole untreated hair follicle.

FIG. 36 shows a higher magnifications of various aspects of an extracted pathogenic whole untreated hair follicle.

FIG. 37 shows a higher magnifications of various aspects of an extracted pathogenic whole untreated hair follicle.

FIG. 38 shows a higher magnifications of various aspects of an extracted pathogenic whole untreated hair follicle.

FIG. 39 shows a peri-thrix picture showing different wavelength and resonance optical contrast aspects to show variances in structure at extreme high magnification without the use of fixing agents or stains and preformed at chair side to the patient.

FIG. 40 shows a peri-thrix picture showing different wavelength and resonance optical contrast aspects to show variances in structure at extreme high magnification without the use of fixing agents or stains and preformed at chair side to the patient.

FIG. 41 is a link which shows standard published pictures of Hamamoto's pathologic lesions.

FIG. 42 show high magnification image using the invention and these images show similarities in structure to Hashimoto's disease. These structures seem are believed to be a function of the co infection and tissue destruction by Aspergillus niger and Borrelia burgdorferi.

FIG. 43 show high magnification images using the invention and these images show similarities in structure to Hashimoto's disease. These structures seem are believed to be a function of the co infection and tissue destruction by Aspergillus niger and Borrelia burgdorferi.

FIG. 44 show high magnification images using the invention and these images show similarities in structure to Hashimoto's disease. These structures seem are believed to be a function of the co infection and tissue destruction by Aspergillus niger and Borrelia burgdorferi.

FIG. 45 is a link to standard published pictures of rheumatoid arthritis lesions.

FIG. 46 show both direct thrix microscopy and peri-thrix tissues. The structures labelled “RA” show similarities to the lesions found in synovial fluid in patients with rheumatoid arthritis. These structures the invention shows appear to be co infection pleomorphic like Lyme disease and fungal.

FIG. 47 show both direct thrix microscopy and peri-thrix tissues. The structures labelled “RA” show similarities to the lesions found in synovial fluid in patients with rheumatoid arthritis. These structures the invention shows appear to be co infection pleomorphic like Lyme disease and fungal.

FIG. 48 show both direct thrix microscopy and peri-thrix tissues. The structures labelled “RA” show similarities to the lesions found in synovial fluid in patients with rheumatoid arthritis. These structures the invention shows appear to be co infection pleomorphic like Lyme disease and fungal.

FIG. 49 show both direct thrix microscopy and peri-thrix tissues. The structures labelled “RA” show similarities to the lesions found in synovial fluid in patients with rheumatoid arthritis. These structures the invention shows appear to be co infection pleomorphic like Lyme disease and fungal.

FIG. 50 shows a link to a standard published pictures of retinal degeneration lesions.

FIG. 51 hows medium magnification using the invention of peri-thrix tissues of lipid and immune cell complex. “R” denotes similarities in pathologic lesions seen in retinal degeneration disease.

FIG. 52 shows medium magnification using the invention of peri-thrix tissues of lipid and immune cell complex. “R” denotes similarities in pathologic lesions seen in retinal degeneration disease.

FIG. 53 shows peri-thrix tissues in different contrast wavelengths with resonance taken using the invention.

FIG. 54 shows peri-thrix tissues in different contrast wavelengths with resonance taken using the invention.

FIG. 55 shows peri-thrix tissues in different contrast wavelengths with resonance taken using the invention.

FIG. 56 shows a link to IDIOPATHIC RECREATIONAL DRUG RELATED LESIONS

FIG. 57 shows a resemblance to synovial fluid lesions in idiopathic drug induced neurological disease.

FIG. 58 shows a SPIROCHETE BACTERIAL/FUNGI SYMBIOTIC RELATIONSHIP.

FIG. 59 shows SPIROCHETE BACTERIAL/FUNGI SYMBIOTIC RELATIONSHIP.

FIG. 60 shows SPIROCHETE BACTERIAL/FUNGI SYMBIOTIC RELATIONSHIP.

FIG. 61 shows a double crab claw structure.

FIG. 62 shows the internal repair structure from early infection of the hair shaft that resembles a double crab claw structure as if two crabs were shaking hands.

FIG. 63 is a link to a standard published pictures of pathologic structures associated with Alzheimer's disease.

FIG. 64 is a link to standard microscopic view of a hair follicle by traditional microscopy.

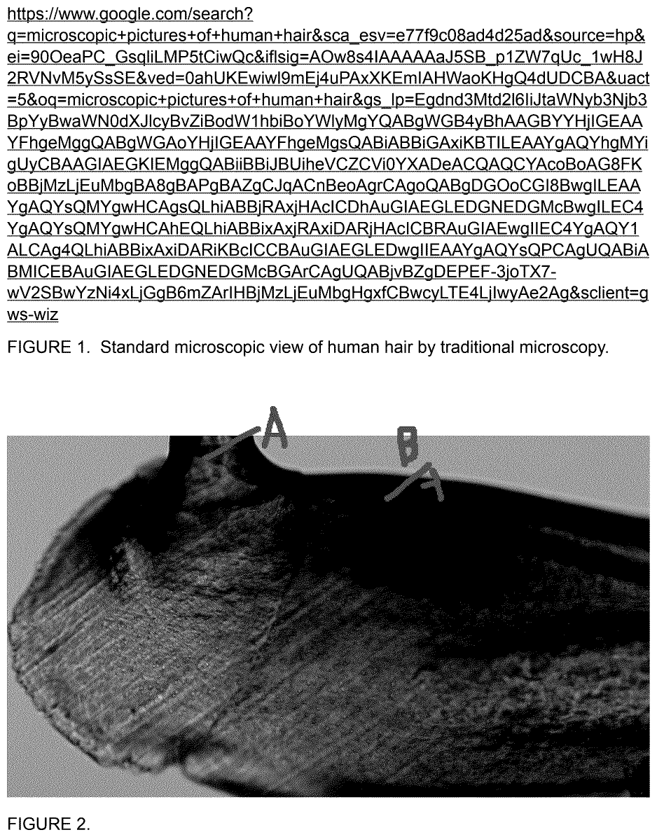

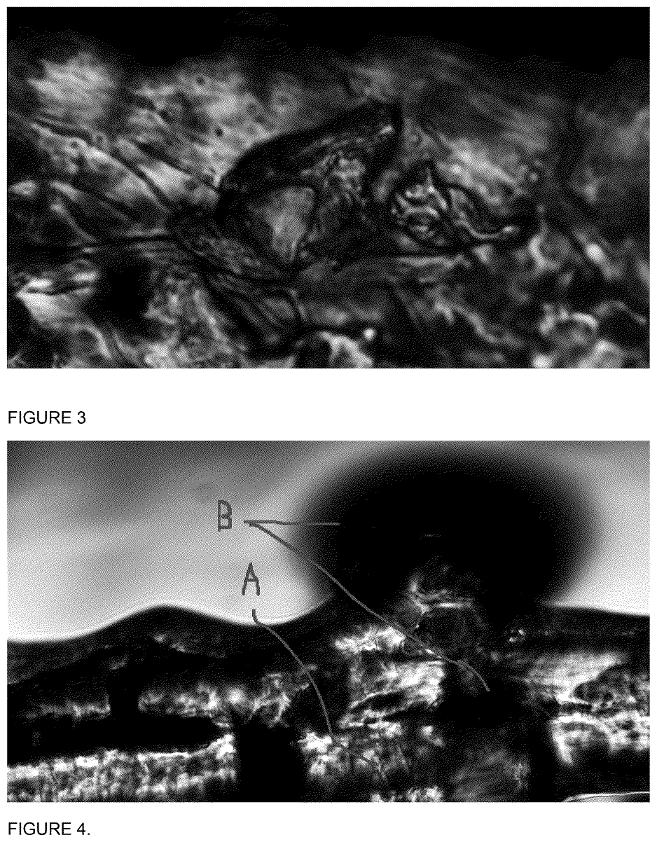

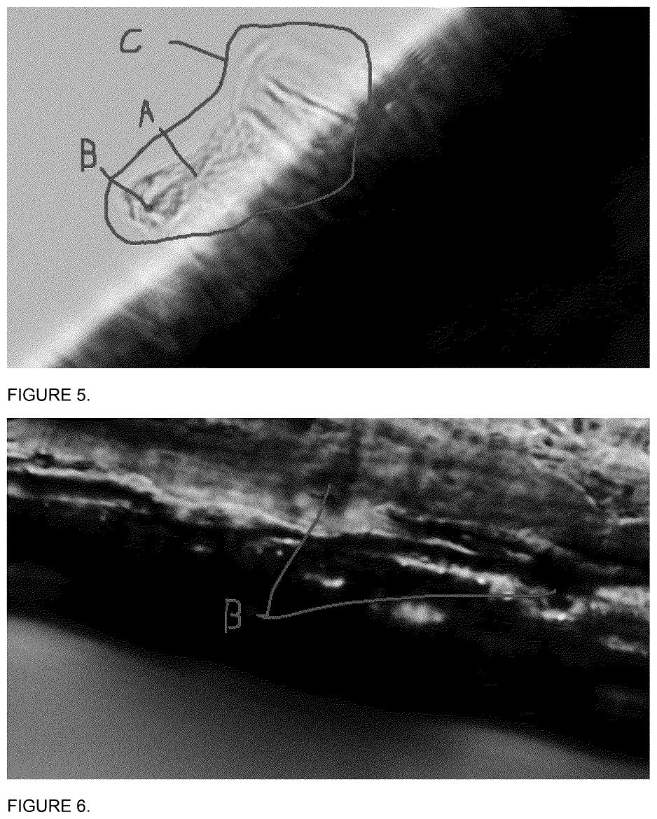

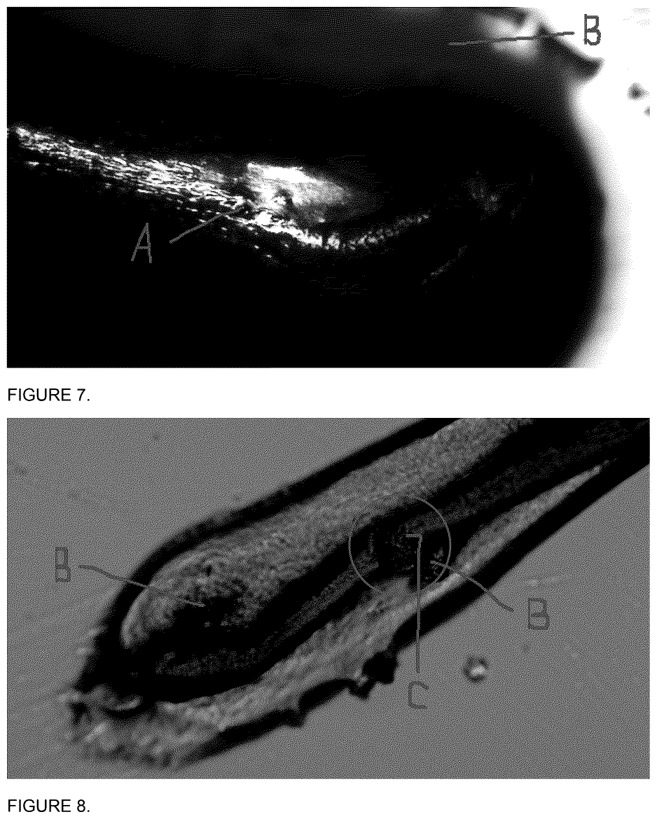

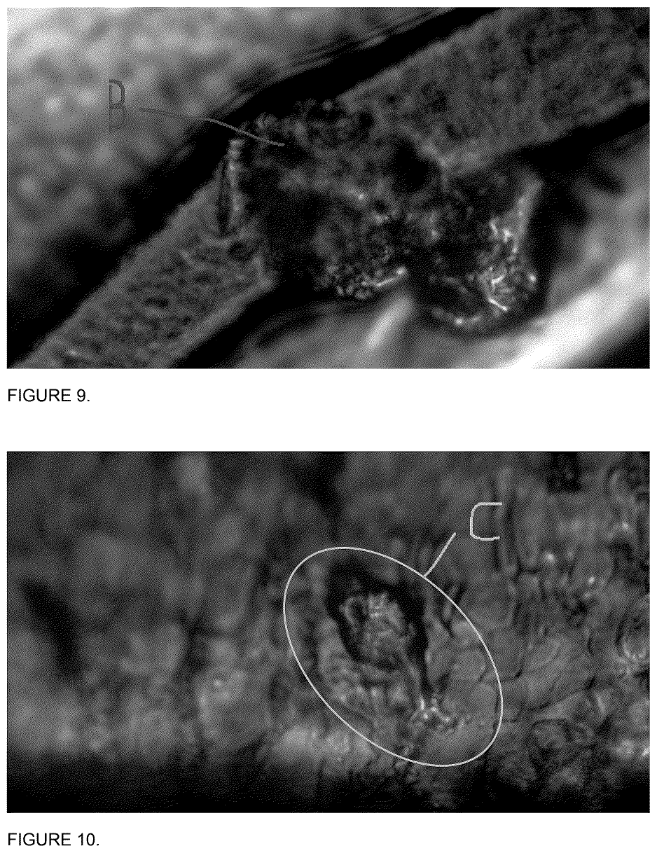

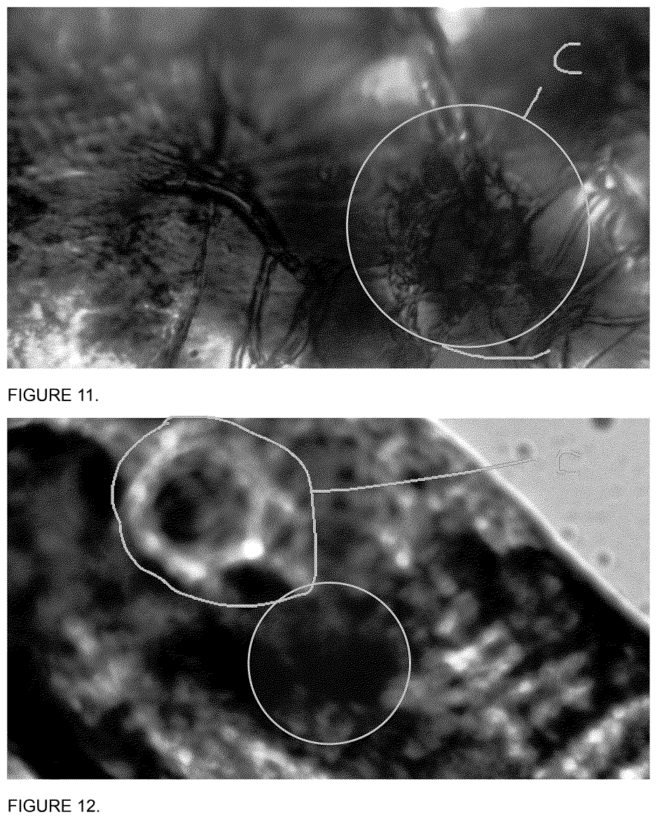

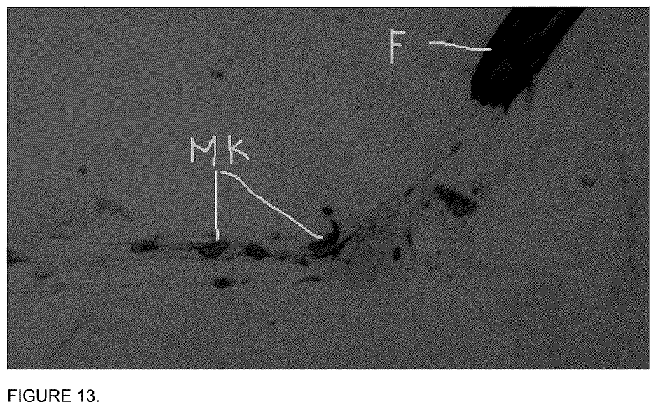

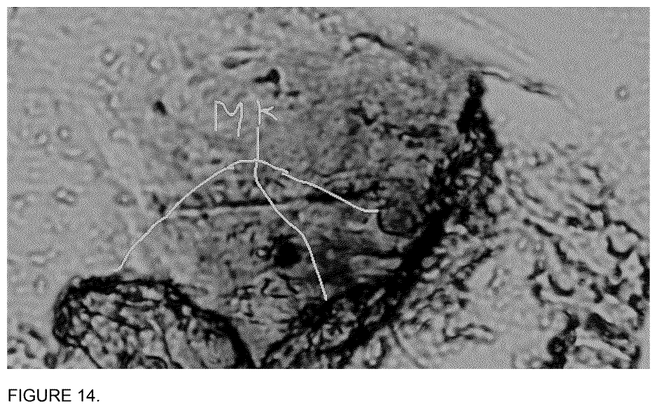

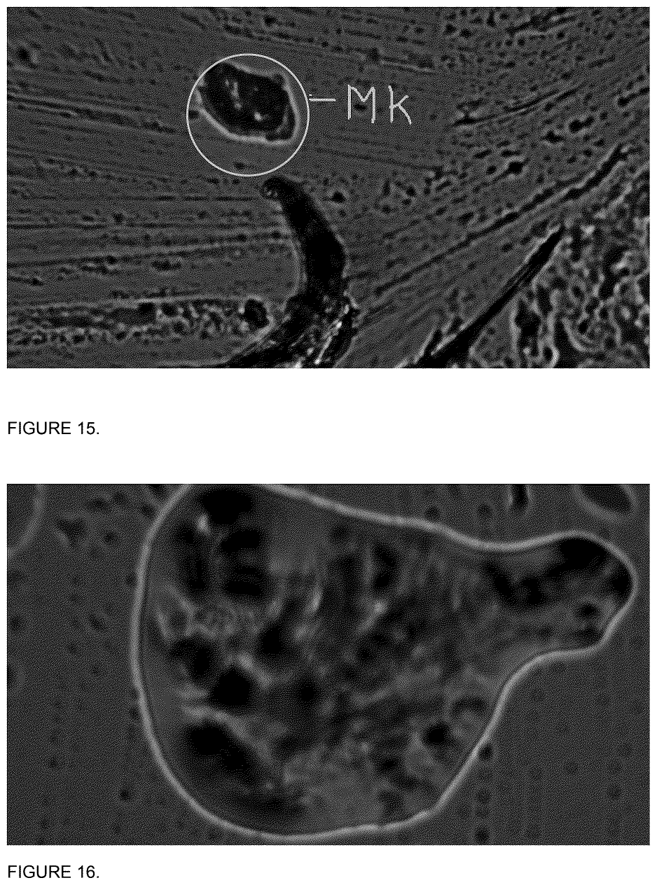

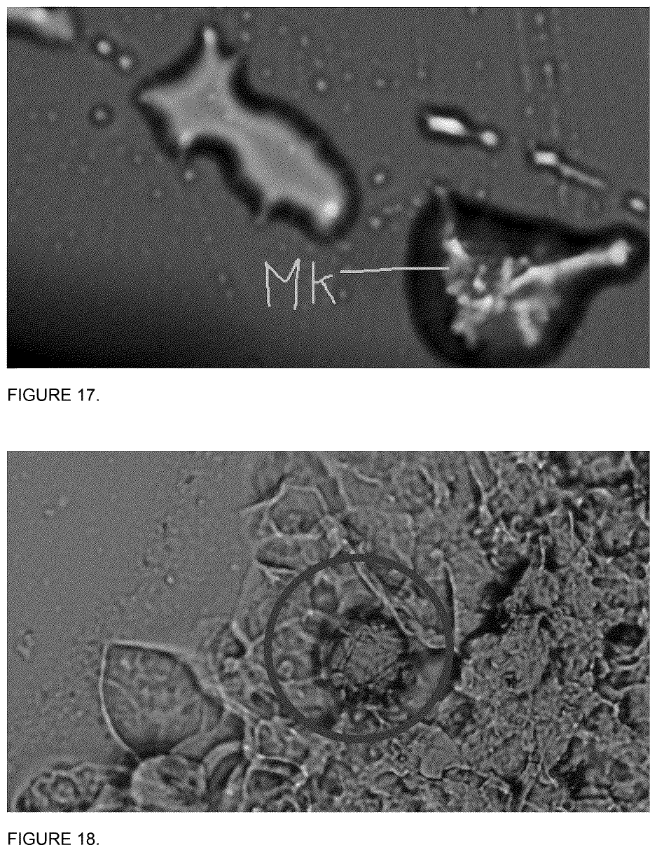

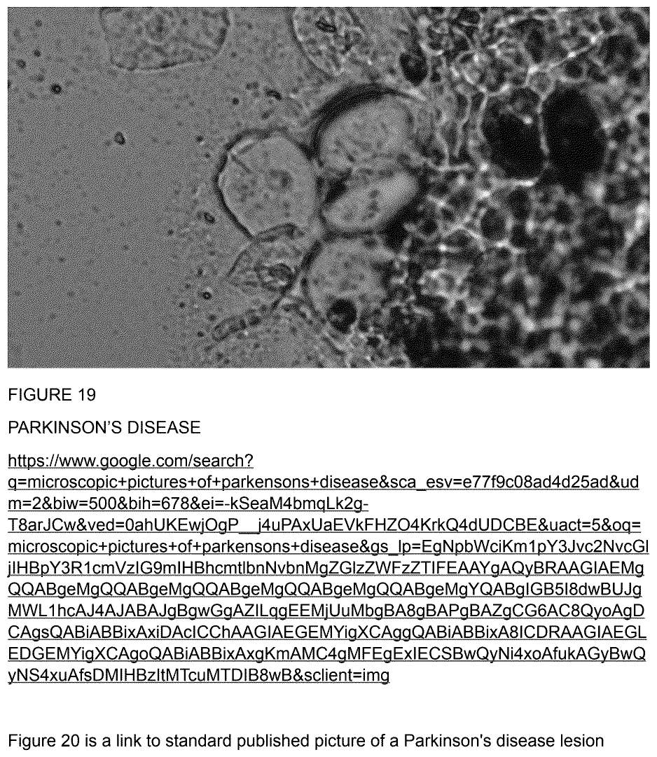

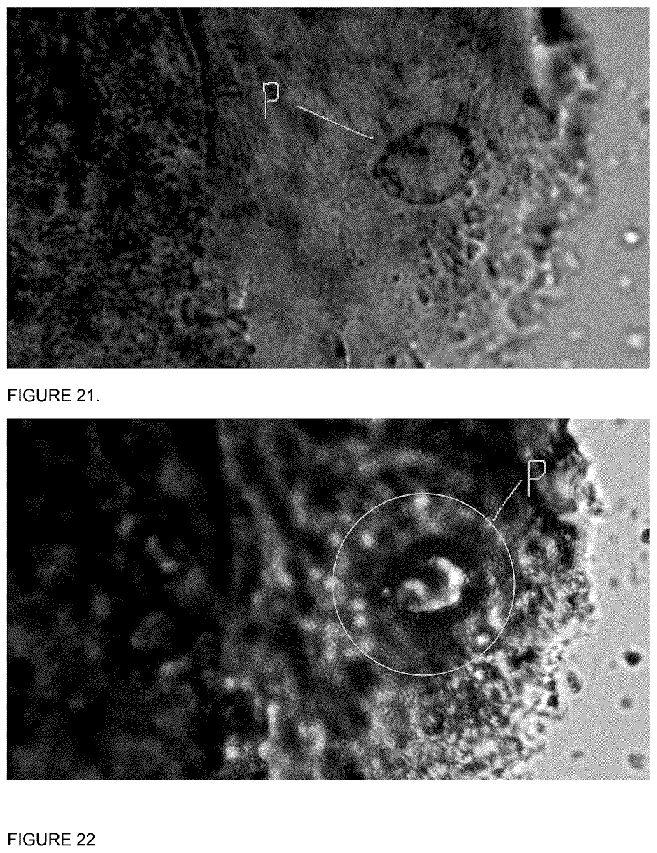

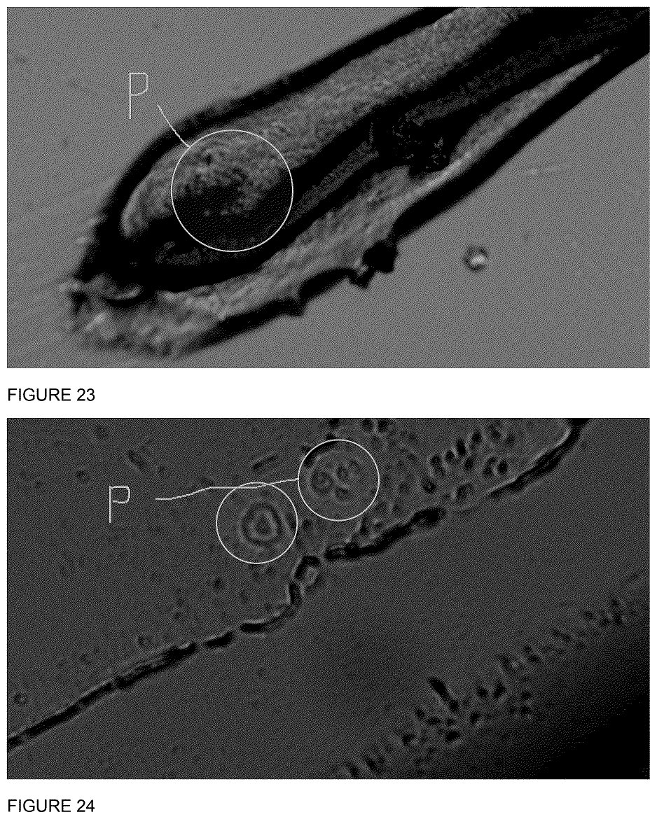

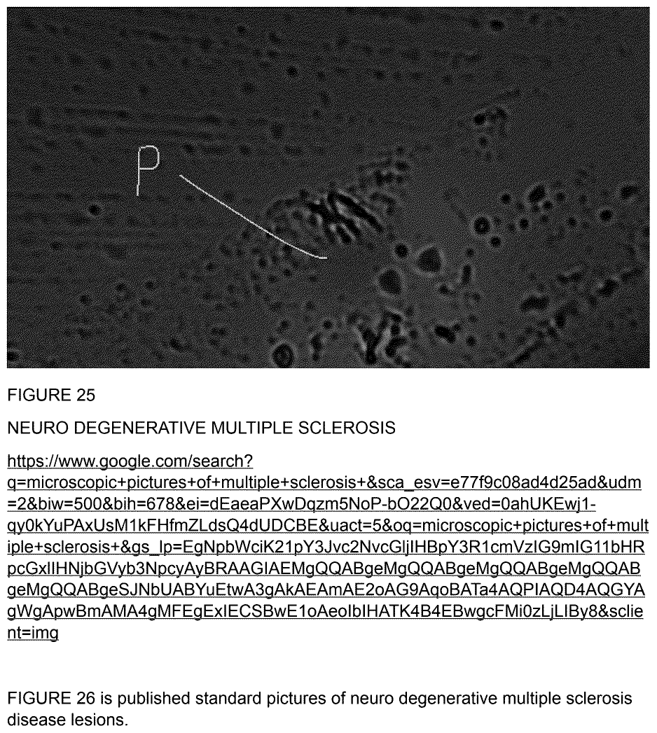

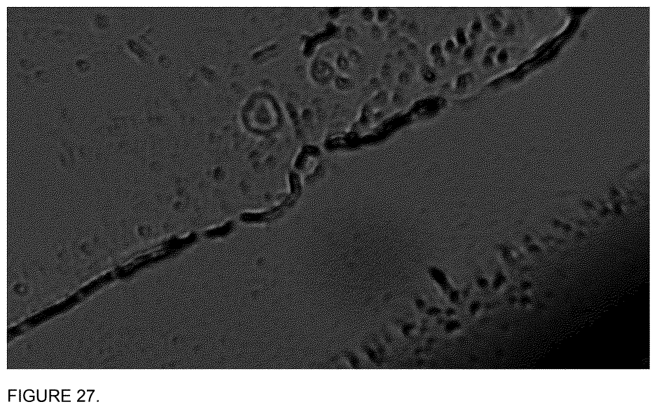

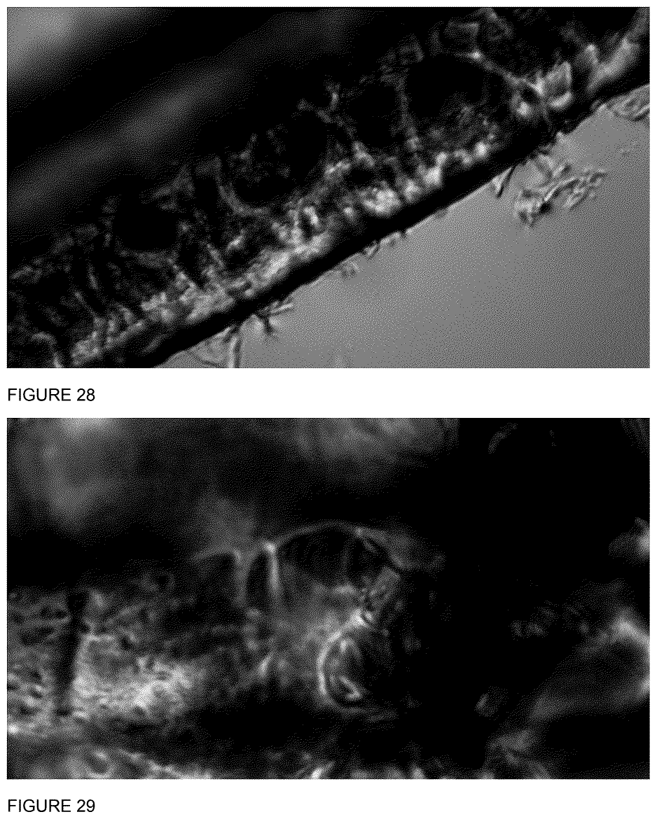

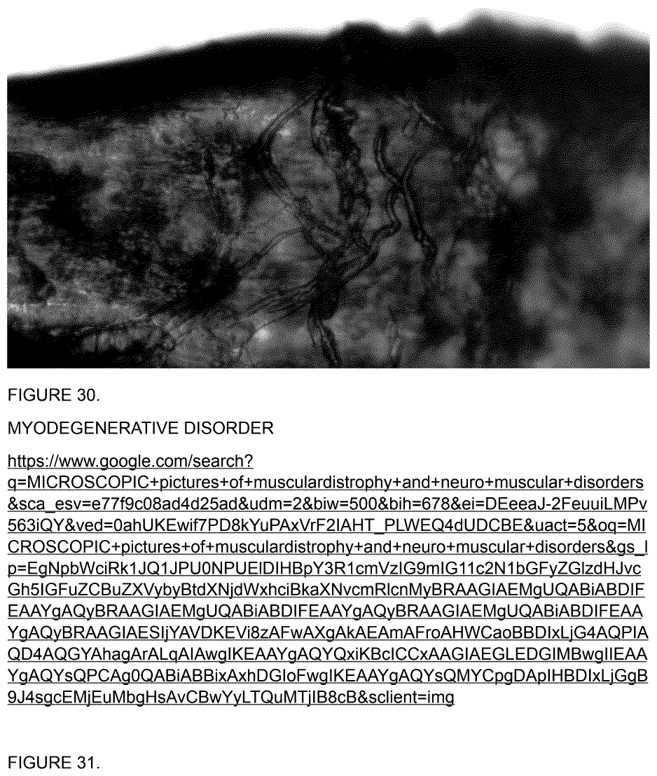

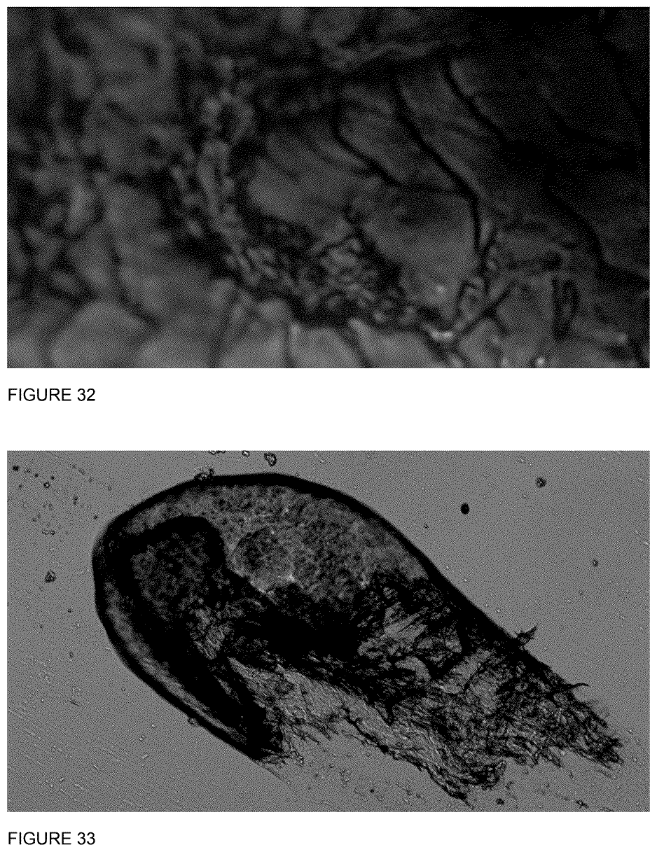

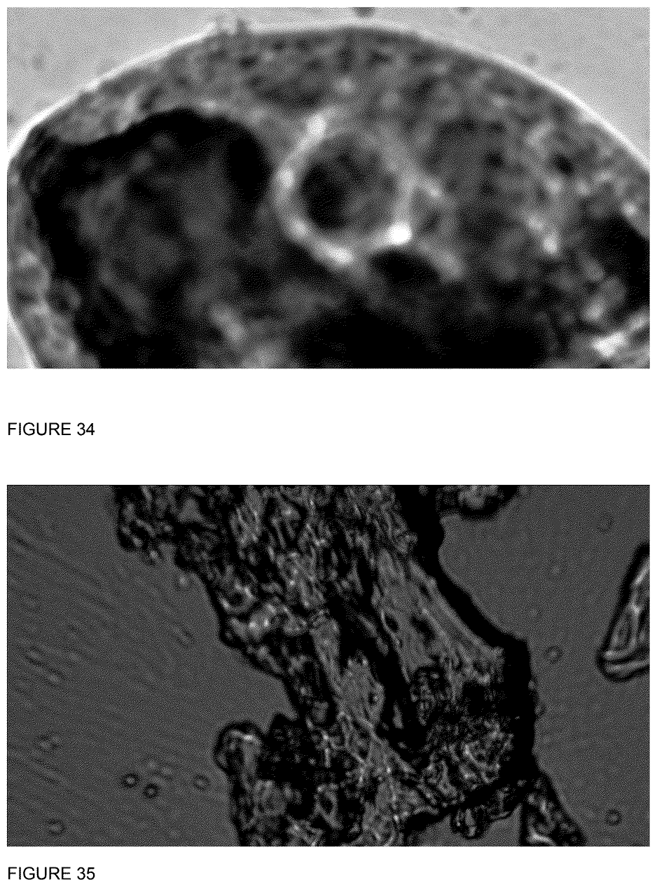

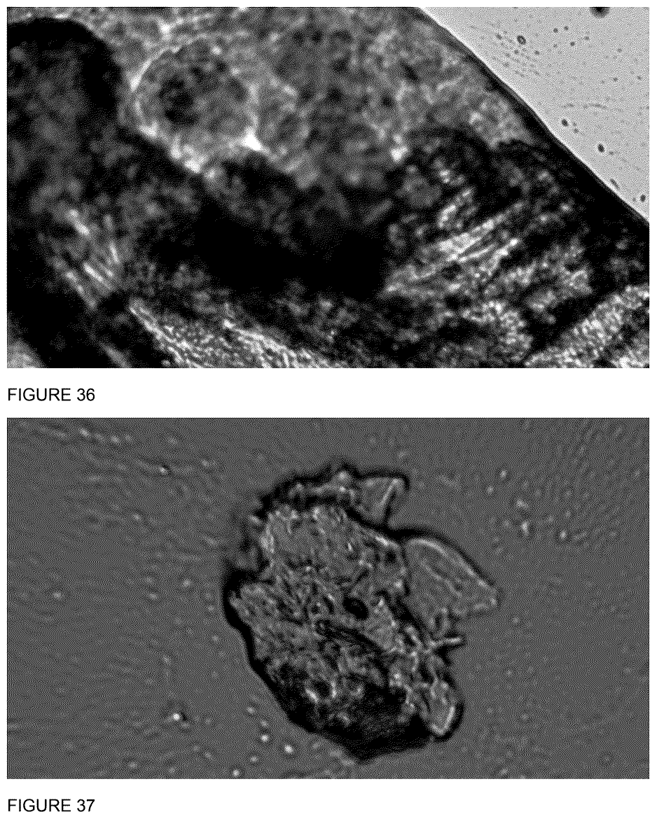

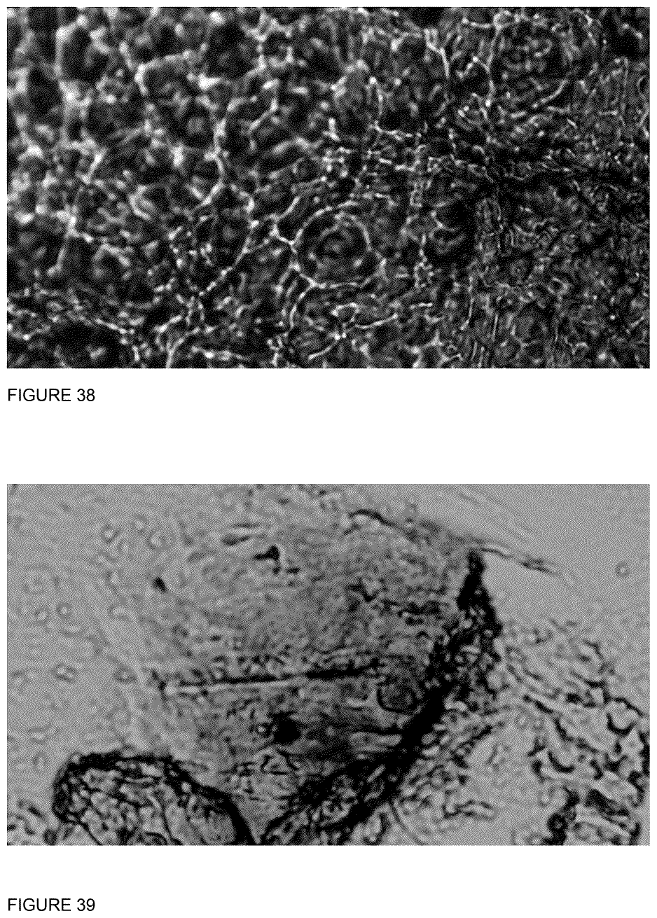

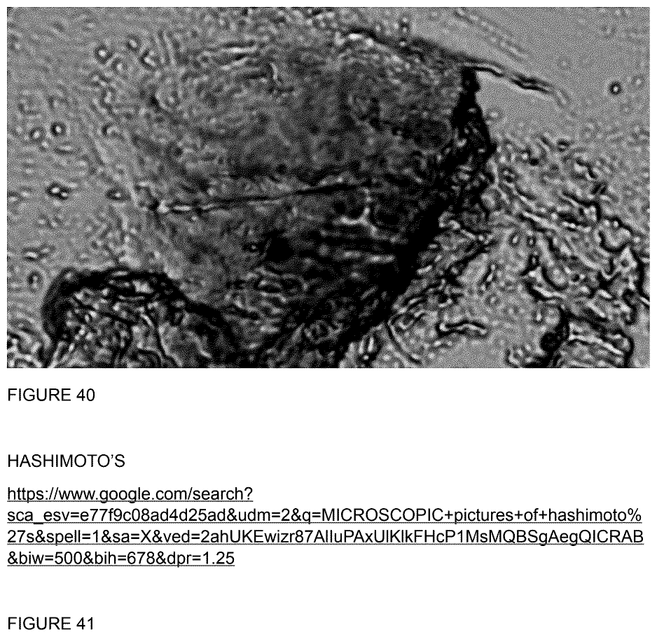

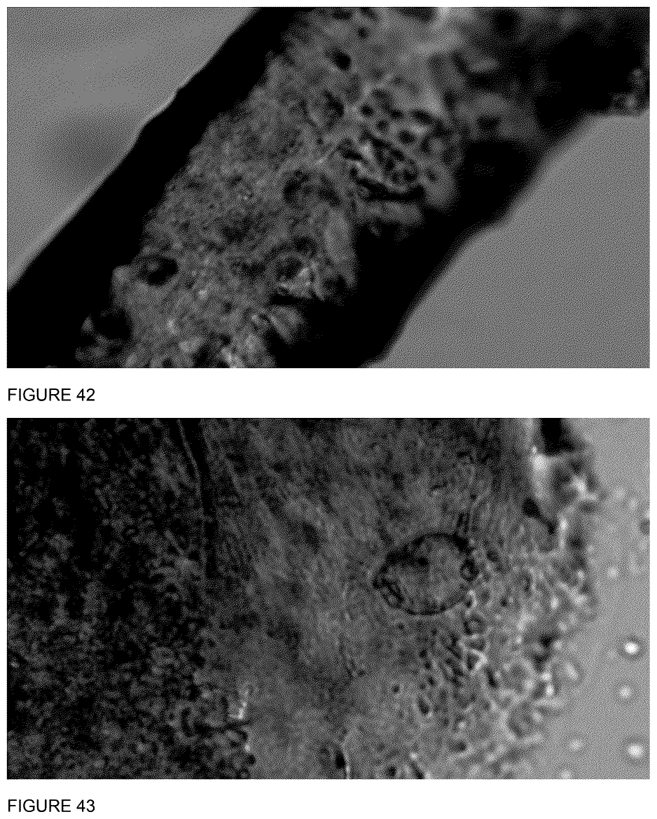

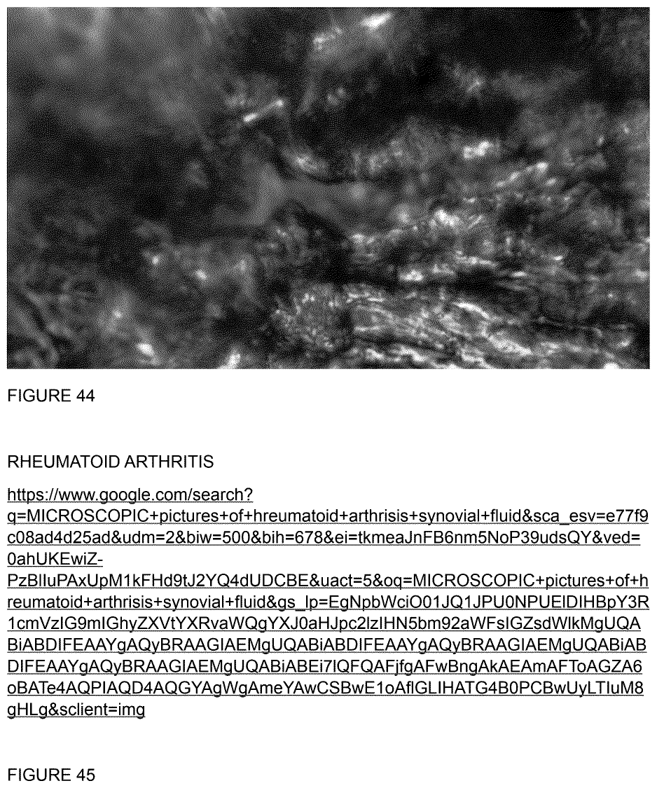

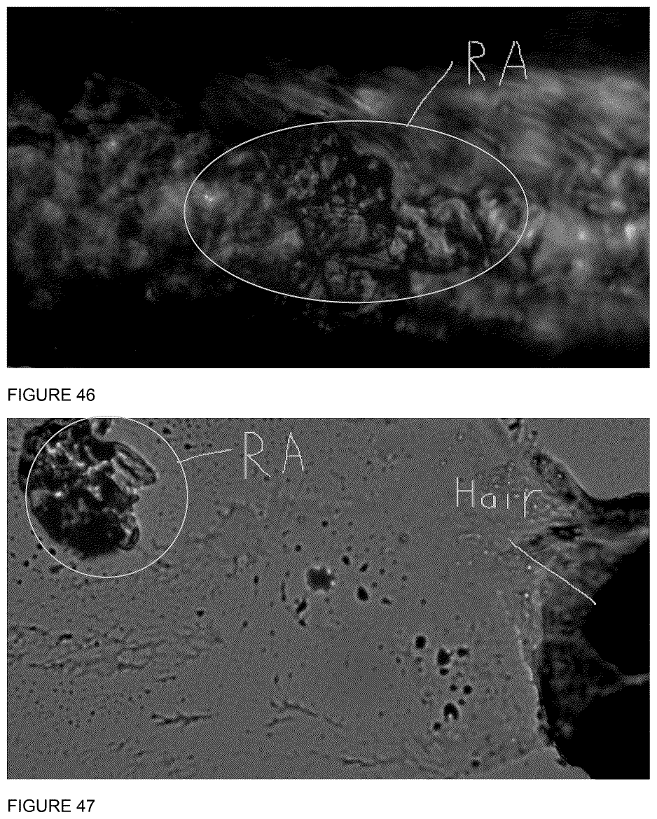

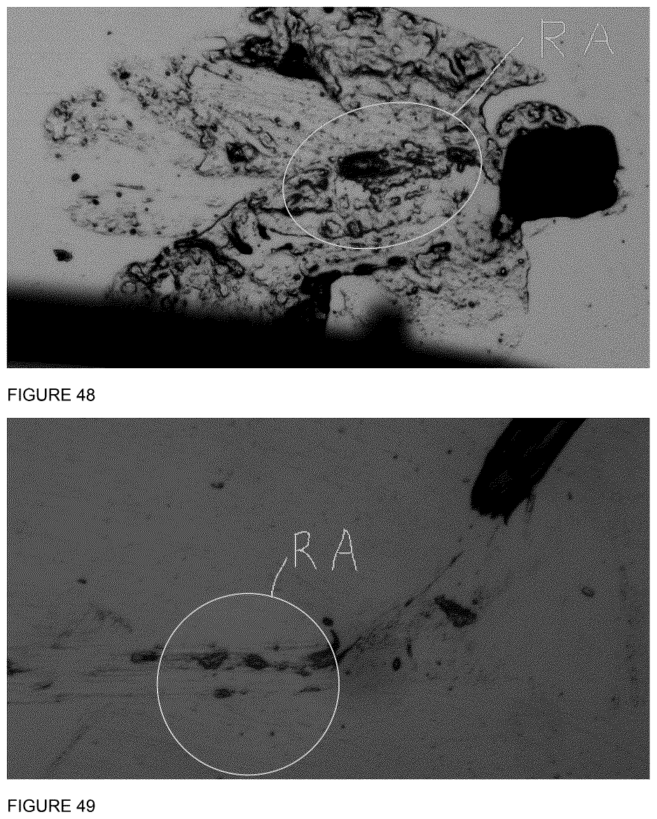

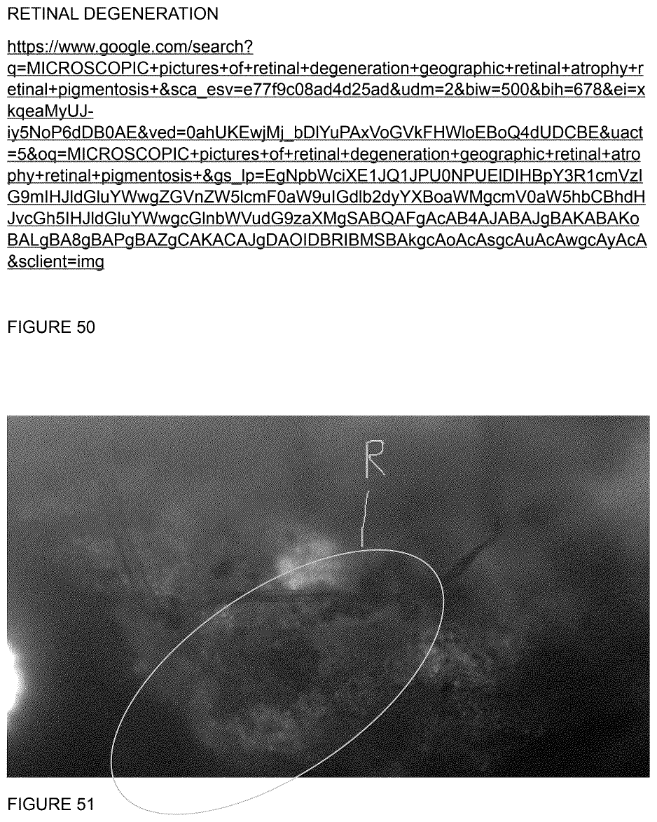

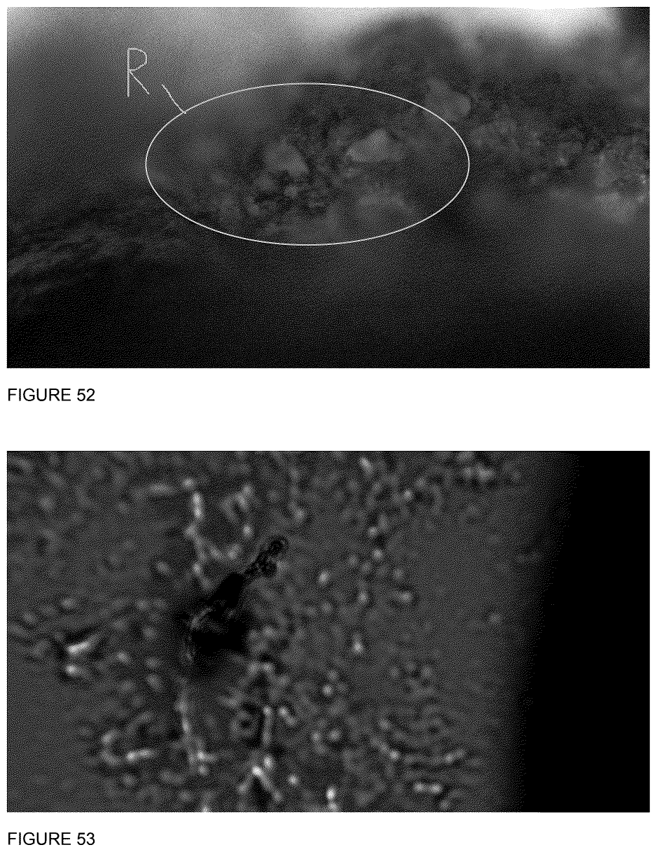

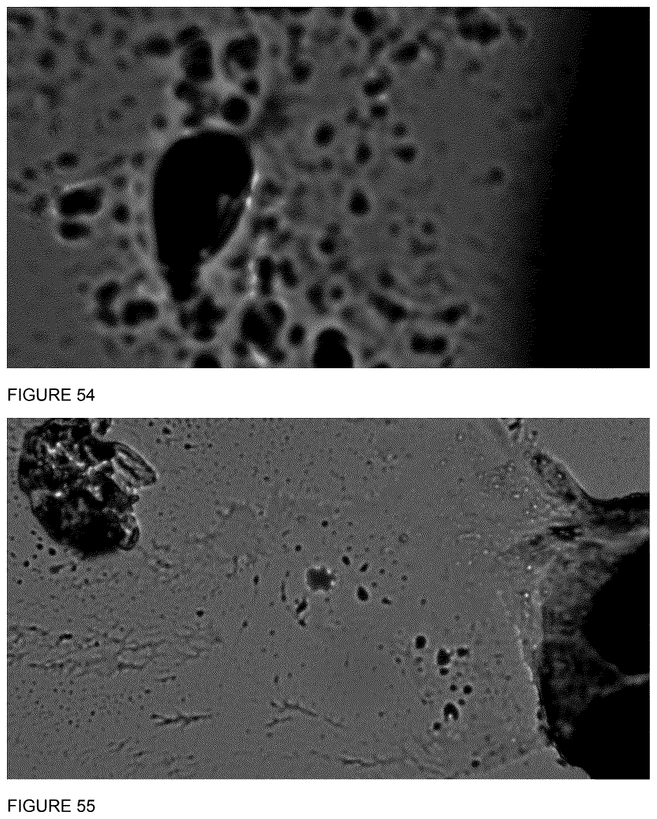

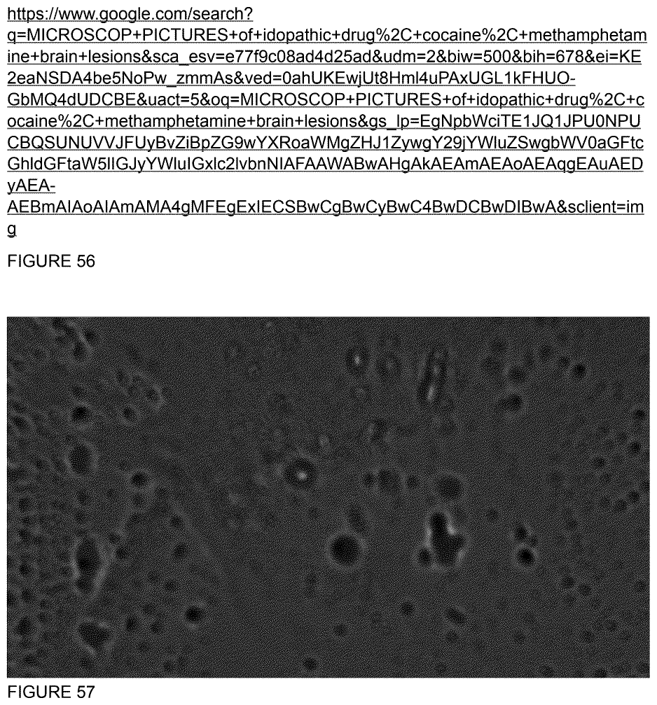

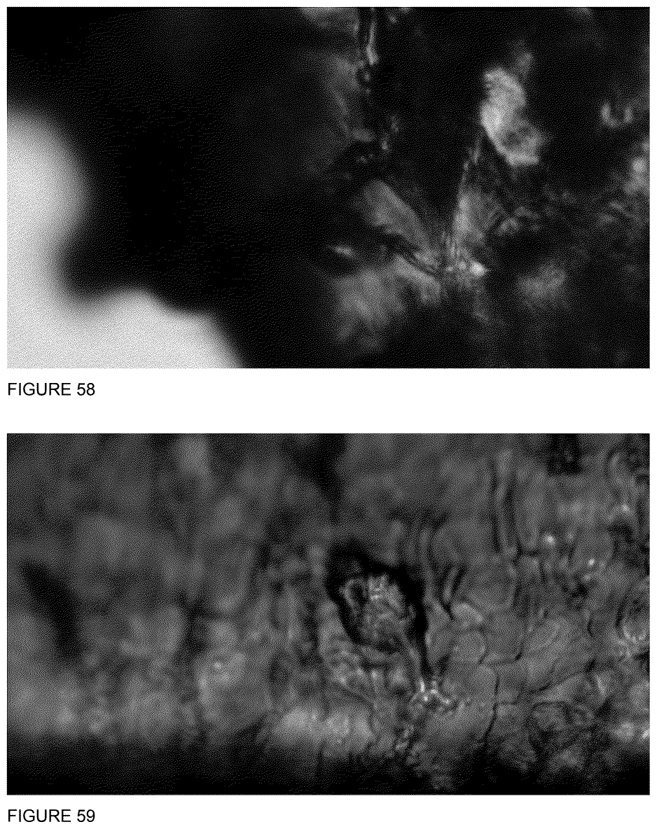

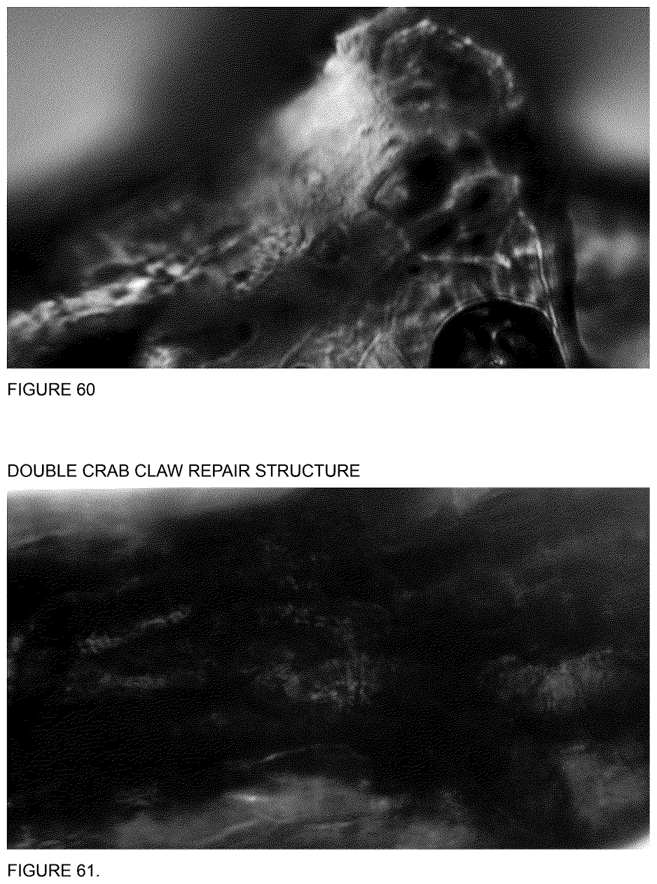

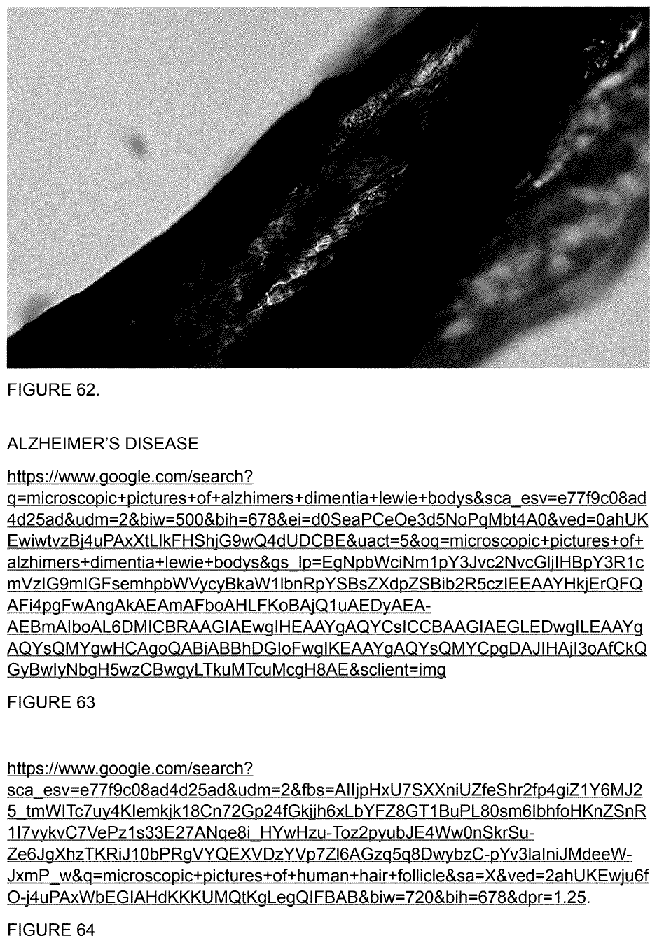

### DETAILED DESCRIPTION OF PREFERRED EMBODIMENT

Referring now to the instant invention, the images in the figures, the instant invention provides that pictures, for example, of hairs 10,12 which each provide an indication to specific state of health or disease correlating to hair characteristics, including texture, color, and structural and cellular changes, and whether organism such as fungi, co-exist to infer underlying health conditions. Ultimately, a goal is in early diagnosis and treating the condition, such as using an anti-fungal to aid in recovery of the condition. The invention provides a method to predict the disease process subject, the method comprising: a. obtaining a biological sample from the subject;
b. assaying an expression level of one or more fungal or bacterial infection expression of structural changes in the biological sample; and
c. classifying the biological sample as from a human and having co-existing bio-organism, such as fungi or bacteria, therewith and by applying an algorithm to the predetermined indicia, thereby predicting the disease of the subject.1. Hair Structure and CompositionHair is composed of keratin, a fibrous protein, and its growth and appearance can be influenced by various internal and external factors. The structure of hair, including its shaft, follicle, and root, can reflect nutritional status, hormonal balance, and overall health. Changes in hair texture, color, and growth patterns have been associated with certain diseases, providing a theoretical basis for using hair as a diagnostic tool.

2. Diagnostic Potential of Hair AnalysisHair analysis has been explored in several contexts:

Nutritional Deficiencies: Variations in hair composition, and disease demise process, which includes factors such as mineral levels, can indicate deficiencies or excess amount of in vitamin D zinc, iron, hormones and B vitamins, and neurotransmitters.

Hormonal Disorders: Conditions like thyroid dysfunction, low testosterone and estrogen and polycystic ovary syndrome (PCOS) have accumulation to fungal or bacterial infection. Toxic Exposure: Hair can store trace elements and toxins, providing insights into exposure to harmful substances.

### Bio-Substances: Co-existence of Bio Substance Provide Indication of Potential Disease. Methods of Hair Analysis

Obtaining the hair at the site of interest on the subject and then placing in a vessel. Use of a compound microscope in oil or non-oil surface vicinity with staining optional for immuno attachment identification, cover slip optional, dark or light field with multiple light source variations, color through red ultraviolet, polarity, disease, intensity, can be some of the factors utilized in the hair analysis to disease on ectothrix and endothrix fungal or bacterial infection. Hair analysis can include chemical assays, such as mass spectrometry and atomic absorption spectroscopy, to detect and quantify substances in hair samples. While these techniques provide detailed information about hair composition, they typically require physical samples to be sent to a complex laboratories rather than images taken on site or simple laboratory setting. Recent advancements in imaging technology, including high-resolution photography and computer vision algorithms, can further assist in this area. The instant invention also employs the image analysis of hair images for key factors, including co-existing bio-organisms. Techniques like digital image analysis can assess hair density, texture, and color changes and cellular changes offering non-invasive diagnostic possibilities.

Studies have examined correlations between hair characteristics and specific diseases. For instance: Alopecia Areata: Characterized by sudden hair loss, which can be indicative of a traditional autoimmune response. Cancer Treatments: Chemotherapy-induced changes in hair texture and color might serve as indicators of treatment effects and patient response and fungal or bacterial induced autoimmune, autoimmune misdiagnosis disease due to the presence of subclinical and preclinical unknown and undiagnosed pathogenic fungi or bacteria.

The hypothesis that a picture of hair can indicate a particular disease relies on the assumption that visible changes in hair can accurately reflect underlying health conditions. While some diseases manifest in observable changes in hair, such as thinning or discoloration, the correlation is not always straightforward. Factors such as genetics, environmental influences, and personal care routines can also affect hair appearance. In addition, it is found that co-existing bio-organism, such as fungi or bacteria, can be an indication of certain disease or the cause. For example, in addition to such diseases as various optical fungal and pathogenic indicators, it may reveal indication of ADHD, Alzheimer's disease, Amyotrophic lateral sclerosis, Friedreich ataxia, Huntington's disease, Lewy body disease, Parkinson's disease, Spinal muscular atrophy, Motor neuron disease, stroke, retinal degeneration, injury, meningitis, AIDS, or Wenicke Karsakoff syndrome and neurodegenerative diseases of neural and melanin tissues. Limitations and ChallengesVariability in Hair Characteristics: Hair characteristics can vary widely among individuals, making it difficult to establish universal diagnostic criteria. Lack of Standardization: There is a need for standardized protocols for capturing and analyzing hair images to ensure consistency and reliability. Complementary Diagnostic Tools: Early hair analysis should be used to develop or warrant other diagnostic methods for more accurate disease detection. Improved Imaging Techniques: Advances in imaging technology could enhance the ability to detect subtle changes in hair indicative of disease. Integration with Other Biomarkers: Combining hair analysis with other biomarkers and diagnostic tools could improve accuracy and diagnostic value. Longitudinal Studies: Research examining hair changes over time in relation to disease progression could provide deeper insights into the relationship between hair characteristics and health conditions. The invention employs a pathogenic analyzer which can be a human optical pathologist and/or computer based device which analysis includes covariance, a statistical method that analyzes the differences between multiple datas criteria while controlling for the effects of at least one continuous covariate. The human pathologist analysis is performed by a trained professional or AI assisted which is trained with a training set including a training sample with a pathology selected from the group including healthy and unhealthy hair images, and any combination thereof. For fungal infection, standard digital processes can be employed with the instant invention optical process. The hypothesis from the instant invention is that a picture of hair can further aid as an indicator a particular disease is supported by some evidence but remains a complex and evolving area of research. The invention is promising for While hair analysis offers promising non-invasive diagnostic possibilities, it is not yet a standalone diagnostic tool. Future research should focus on refining imaging techniques, standardizing analysis methods, and exploring the integration of hair analysis with other diagnostic modalities to enhance its clinical utility. Case Example. Patient observed. Hairs were taken form the face and neck in the areas of inflammation and irritation and pain. Later in the process, hairs were taken from other areas of the body including abdomen chest arms and legs due to the facial connection. The initial impression of a diagnosis was achieved in 3 minutes after the first hairs taken and examined under a compound microscope. The hairs were extracted and immediately placed on a microscope slide with no cover slip. Very little pain or no pain was noted during the hair extraction. No anesthetic was used. Immediately after the extraction the area was felt to be relieved of itching irritation or pain. No cover slip was done not to damage or deform the hard tissue hair and soft tissue present that was removed with the hair form dermal perifollicular peri hair tissue. No stain was used to view the samples. Approximately 200 samples taken and evaluated over the course of approximately one month. Digital display microscope with direct digital video and picture capability saving to an internal SD card. All images saved on the SD card came directly from the samples viewed by the microscope. Different settings were used such as light variation in color and intensity to view the samples allowing for different structures and depths of field to be viewed of the 3 dimensional sample. Focus was used to examine different layers to assimilate a mental 3D picture. This was portrayed in several videos saved showing sample depths at different focal points. Much of the sample examination was done using a dark field environment was found to be helpful in viewing the samples. Magnifications used were between 40× and 2400× although higher magnification proved difficult with the thickness of the hair a few high power images were obtained and extremely helpful in understanding the pathology. Low magnification was also extremely useful in determining the initial probability of pathology present due to the unique abnormal shape and structure of the hairs. Red and blue light filters and other filters and accessory light sources seemed to be most helpful in viewing the fresh tissue samples.

### Findings Low Magnification Gross Examination

Hairs are dark in nature with occasional translucent areas. Barb like projections noted on the hairs through the length of the hair and subdermal and epidermal portion. Some hair samples have a thick coating of soft tissue along the entire subdermal and epidermal portion of the hair.

A white bulb approximately 1 mm in diameter is present on the outermost epidermal portion of several hair samples. The epidermal soft tissue attached and the white bulb areas are readily visible with no magnification. The abrupt bends in several hairs is also visible with no magnificationHairs have abrupt bends and kinks and twists in them and multiple bulbous regions but most notable in subdermal portion. The dermal layers are estimated due to no dermal tissue by the length of the hair sample extending into the skin. Some samples show multiple hair filaments emerging form one root area. Generally one filament is average size of the samples and one is much smaller approximately one half to one 10th the size of the larger hair in the sample. The smaller hair filament is generally more curved and spiral and more translucent and very thread like. The larger main hair seems to be stiff ridged except for weak points where bends occur. Most are of a single sweeping curve or relatively straight.

### Medium Magnification External Structure of Hair Sample

The barb like extensions appear to have active dark brown or black in color cells on the ends. These seem to be dysplastic cells and likely a mechanism for proliferation or metastasis Filament thread like structures noted arising directly from several main body hairs. The filaments seem to be small hair like threads with keratin fibrous internal structure. The same active dysplastic keratin melanocytic cells and pigment can be observed on the barb ends of the small filament structures growing off or around the main hair. Within the subdermal soft tissue surrounding many of the extracted hairs. keratin melanotic hybrid dysplastic globular groupings of cells can be seen not directly attached to the main hair structure or the secondary filament thread like hairs. It appears these groupings of cells have dislodged and are roaming free and entering the dermal and sub dermal region The sub dermas portion of the hair is up to 6 or 7 mm in length. The longer of the subdermal hair structures seem to be bulbus at the subdermal terminal end and do not have the typical bi keratin root associated with normal hair connection to the follicle.

INTERNAL STRUCTURE OF HAIR SAMPLE Multiple sections of keratin looking strands with crab claw like ends but not attached to any other keratin strands. Multiple breaks in keratin structure, breaks are associated with the barbs on the outside of the hair structure mostly. Dark black hollow like vesicle shaped voids in the keratin strands and medulla portion. First impression is this is likely and immune response of unknown cell type such as macrophage or lysosome activity. Dark cellular and extensions seem to be present on the outside of the hair both sub dermal and extra dermal portion of the hair.

Dark black circumferential bands noted around the hair. These seem to be multiple fungal or bacterial colonies lined up.

Fungal or bacterial extensions seem to be associated with the breaks in the keratin structure of the hair.

Circular voids present within the keratin structure of the hair. In these voids exist a single or small group of 2 to 4 cells. The circular structure has the appearance of almost an egg or bud with a new cell type this seems to be fungal or bacterial growth and reproductive in nature.

In some areas the dysplastic keratin looking fungal or bacterial cells seem to be escaping the egg as a new type of cell or group of cell structure and attacking other normal keratin structure causing it to disintegrate. The white bulbus circular structures at the outer subdermal portion of the hair samples previously noted on low magnification and gross evaluation are surrounding a melanotic enlargement complete grouping of cells attached to the hair body. It seems likely that this white portion of tissue is a collection of immune cells.

On higher magnification it seems very similar to a collection of lymphocytic cells and seems highly organized and coating the melanocytic non uniform cells this appears to be the immune response attempting to attack the fungus or bacteria.

The structure of the melanocytic grouping of fungal or bacterial cells is random and non uniform and jagged in nature. Protrusions are noted in all directions and some extending through the lymphocytic layer. The end portion of the extensions seem to have the same dysplastic keratotic melanotic cells likely for proliferation of the dysplastic complex. The internal structure of the hair at this complex is highly unorganized and has the appearance of recent destruction of the keratin filament structure. Many new crab claw like filaments noted and intra keratin egg like structures with dysplastic keratin cells inside are noted in higher concentration than other areas of the hair.

### High Magnification Internal Structure

Melanotic fungal appearing cells with long tentacle like protrusions extend into the keratin structure of the hair. The protrusions seem to be present where the egg like vesicle containing the dysplastic keratin like cell groupings. White filament or snake like filaments within the melanocyte protrusions can be seen termination into the egg structures and healthy keratinocytes. Unknown clear some almost white looking melanocytes or dendritic cells or non melanized fungal cells are also noted with the same projections going into the internal keratin filament structure of the hair. The sections of hair or abrupt bends noted on low magnification are noted on high magnification to be connected strands of keratin filament. The connections are described as crab claw like ends where pincer claw is grabbing pincher claw as if two crabs are shaking hands or holding onto on another in a line. Some of the claw like keratin strands have a claw pincher structure on both terminal ends and some connected at both ends and some not. There is not a definite uniform structure among the samples and general structure among disorganized filaments and connections seem to be somewhat random in nature possible causing bends in some and not in others.

Dark to black non uniform groupings of fungal or bacterial melanocyte-like cells noted within the hair keratin structure and mostly present at the areas of seemingly destroyed keratin filaments.

### General Impression

The observations above seem highly non normal and likely a dysplastic condition of fungal or bacterial infection possible leading to melanoma or keratin based pre squamous cell carcinoma or other cancer type disease process due to localized immune suppression by the fungus or bacteria. Destruction is noted to be extreme and evident through the sample group. Melanoma starting form a hair follicle has been reported to have a possible association with squamous cell carcinoma by NYU School of Dermatology.

A fungal or bacterial type growth process is possible involved in the white extensions of the fungal cells that are immature and the unknown white or clear dendritic type cells as these extensions have axon like neural cell characteristics. At this time it is simply a rough conclusion. Dysplastic neural crest cells are a possible etiology to the dendritic melanotic keratotic cell mutations. The possibility of dysplastic neural crest cells using the fungus or bacteria as a target for immune response programming seems plausible also but is simply and educated guess off of observations.

### Cation Affinity

It is observed that the fungi appears to thrive in the presence of cationic material, such as iron.

### Conclusion

The samples taken seem to indicate a disease process of peri-thrix and internal endothrix fungal infection and possible indicator of pre melanoma or pre kerato cancerous type of condition. The cells seem highly mobile and metastatic in nature and multiple levels of dysplastic groupings associated with distance from the center of the hair. Preclinical and subclinical infection by fungi and other pathogenic agents such as viral secondary infections to immuno suppressing such as shingles where standard blood and pathological tests fail to identify infection on early formation. This delay in current methodology leads to the progression of fungal and other infections into systematic degenerative disease. The invitation provides an indication for inclusion polymyosis dermatomyosis, polymyosis and other neuro muscular and neuro degenerative diseases along with secondary cancers and viral infections. TREATMENT

Antifungal or antibiotics and at low dose reinforces the diagnosis of fungal or bacterial infection. However, treatment varies depending on the various factors of the subject. By using antifungals and/or antibiotics the recommended dose may vary but the duration likely will remain extended due to the ability of this fungus or bacteria to live within slow growing hairs.

Applicant notes the following. Lyme Disease (bacterial infection of Borrealia burgdorferi) is a debilitating pathogenic disease affecting millions of Americans and countless other people around the world. The pathogenic bacteria Borrelia burgdorferi that causes Lyme disease exists in multiple forms. I serum it exists as Round Body Morphology, Biofilm like colonies, has a flexible cell envelope and form a Round Body structure and a cell wall. Spheroplasts, protoplasts forms of Borrealia burgdorferi lack a cell wall. The L-forms Propagules, cysts have a cell wall but the cell wall is a glycoprotein matrix rather than a traditional cell wall or peptidoglycans. To further complicate the diagnostic process the Borrealia burgdorferi is pleomorphic and changes its morphology depending on its environment. These structural variations include ling filaments. Spral shape, Round Bodies and Spheres This pleomorphic variance in addition to the multi species co—infection of a fungal organism such as Aspergillus niger and multipleospeciesmorphism of anatomical structure makes traditional microscopic identification with staining extremely difficult if not impossible. This invention test intends to easily, early and rapidly identify this bacterial, fungal, and bacterial fungal co infection no matter what morphology it takes in order to treat the patient aggressively.

To further explain why this test is unique and necessary consider the following. The glycoprotein matrix of the Borrealia burgdorferi cell wall or cell membrane is an energy production complex that fuels flagella to make the organism highly motile and highly virulent and highly pathogenic. The theory presented here to consider is that this energy complex and associated factors attracts the fungal component for example here we use Aspergillus niger but another fungi or radiotrophic fungi may be involved. These melanotic fungi absorb sugars and (alpha radiation when present from dissolved radon) and create an additional glycol electron energy complex. The alpha radon does not appear to be necessary to form the symbiotic co infection but it does seem to provide energy for a more virulent progressive infection that likely leads to more disabling neuro and neuromuscular and autoimmune disorders. This complex once formed and mature feeds in a parasitic manor on the muscular and neural tissues and destroys these tissues. Many of the same amino acids like tryptophan and other related factors to make neural transmitters like dopamine and melatonin also can be use to make fungal melanin. The fungal portion of this pathogenic complex seems to be responsible for the dopamine neural destruction component. The bacterial component seems to be more responsible for motility and spreading of the infection deeper into tissues but also may provide tissue destruction. This inventions shows these structures exist in the hair and hair structures shown if the attached figures.

Both fungi and Lyme disease produce an immuno suppressive halo around them but in different manors. Therefor treating both infections and the common connections between the infections, that is the glycoprotein matrix and triad of treatment is this theory of paramount treatment from the diagnostic test invention stated.

The inherent resilience of this bacterial fungal co-infection complex makes identification and treatment difficult. This test will allow for both early identification and early treatment of both organisms at the same time to rid the patient of pathogens and allow healing to begin. The final goal of this test is to eliminate neuro and muscular and neurodegenerative and autoimmune diseases altogether by identifying the pathogens early and treating early to prevent disease. Once this process is established establishment of a vaccine should be possible to prevent any infection whatsoever and render this invention to be a diagnostic aid to ensure the functioning of the vaccine.

Now we will consider the contagiousness pathogenic nature of lime disease of the current understanding and how this novel test can allow for a new understanding and treatment.

Traditionally Lyme disease is considered a localized skin infection from a tick bite. The iconic “Bulls Eye” rash shown in every dermatology text book. The current concept is that that is a tick bite site. And this is likely true. But many patients that are diagnosed with latent extensive Lyme disease do not remember getting bit by a tick or having such a rash. The current medical model thought is ‘’ will you got bit by a tick but you did not know it and the rash was small and you did not notice it or there was not rash. In this invention theory we consider that the patient may not have been bitten by a tick and contracted Lyme disease based on the findings of this test.

The bacteria that causes Lyme disease lives inside the tick and in the saliva of the tick and tick saliva is composed of glycoproteins similar to human saliva. These glycoproteins if tick or human or other animals may allow the Lyme bacteria to exist in an oral pharyngeal environment. Thess bacteria may then be able to penetrate the oral pharyngeal mucosa and enter deep into the tissues as other similar spirochete bacteria. Some may enter the blood stream and some may gravitate to the hair follicles of the skin associated with the close contact to saliva or mucus. This Lyme disease bacteria then meets up with the fungi such as Borrelia burgdorferi and the symbiotic co infection relationship begins and the subsequent co infection spreads into the tissues and blood stream. This spreading and existing of infection undetected is promoted by both species ability to supress the localized immune system in two different ways. this invention will test for pathology at the absolute beginning stage of this co infection of the hair follicle to allow for early diagnosis and treatment of neuro muscular neurodegenerative and autoimmune diseases we know today.

In many nursing and extended care facilities neuro degenerative disorders seem to be spreading among susceptible residents. This invention test would aid in showing early this infection spreading in a closed facility. Finding this disease process early would greatly benefit the lives of many elderly people. This invention test can also be used to study animal models of transmission and transmission by saliva and mucus from animals to animals and animals to people and people to people in a variety of transmission modalities.

In connection with the use of the optics for the instant invention, the following is noted. A surface-mount optoelectronic package can be employed which comprises
a. an aluminum nitride (AlN) submount disposed within a package body having external pads configured for solder reflow;
b. four edge-emitting laser dies attached to the submount and individually addressable, the laser dies having nominal center wavelengths of about 460 nm, 488 nm, 522 nm, and 632 nm (each ±5-10 nm); and
c. an optical redirection/combination module positioned to receive light from the laser dies and provide a single output beam path or a co-boresighted bundle,
wherein the optical redirection/combination module comprises at least one of: a planar lightwave circuit (PLC) with an on-chip combiner and vertical out-coupler, a micro-mirror, a diffractive or metasurface coupler, a total-internal-reflection wedge, a fiber with an angled-polish reflector, or a micro-prism; and
wherein the package is configured for bio-fluorescence excitation via temporal and/or spatial multiplexing of the four wavelengths.

The optical redirection/combination module is a PLC implemented in silicon nitride or doped silica and includes a 1×4 or 4×1 MMI or directional-coupler network to combine the four wavelengths into a single waveguide and a grating coupler or etched 45° mirror to emit substantially normal to the PCB. The multiplexing is temporal, with per-channel gating ≥10 kHz and a monitor photodiode for pulse-to-pulse normalization. The multiplexing is spatial, with co-boresighted emission axes within ±2° and optional micro-lenses to condition fast/slow-axis divergence to ≤5° FWHM.

The package can further comprise an optical fiber interface (ferrule seat or receptacle) to couple the combined output into a 200-400 μm, NA≈0.22 fiber and a sealed lid with an AR-coated optical window and an interlock/ESD network on the submount.

The AlN submount includes thermal vias and an exposed thermal pad; the package achieves a junction-to-ambient thermal resistance ≤35° C./W on a 2-oz Cu test board. The four wavelengths are selected to excite respective fluorophores including DAPI/Hoechst (˜460), GFP/AF488 (˜488), FITC/AF520 (˜522), and Cy3-class (˜632) with appropriate filter sets. The PLC includes on-chip taps and photodiodes to monitor per-channel power and a feedback controller interface.

A multiplexed fluorescence excitation source, comprising the package of any of claims 1-9 mounted on a PCB and optically coupled to a microscope, micro-fluidic device, or flow cytometer. The method of operating the package contemplates selecting one of the four wavelengths or time-interleaving two or more wavelengths, and normalizing intensity using feedback from per-channel monitor photodiodes to maintain constant excitation irradiance at the sample plane.

While the invention has been set forth, it is submitted that the claims which follow should be afforded reasonable scope to cover modifications, derivations and improvements.

What is claimed is:

1. A method to identify a condition in a hair sample, the method comprising:
(a) obtaining a biological hair sample from a subject;
(b) assaying an expression level of one or more element in the hair sample and providing expressed element data;
(c) obtaining image data of the hair sample and generating image data; and
(d) employing a pathogenic analyzer which predicts whether the hair sample comprises at least two elements, thereby predicting one of a healthy condition and an unhealthy condition.

2. The method of claim 1, wherein said algorithm classifies unhealthy condition with associated with a pathogen and a predetermined disease.

3. The method of claim 1, wherein said hair sample is a hair from diseased tissue area.

4. The method of claim 1, wherein said hair sample is a hair from diseased tissue area.

5. The method of claim 1, which includes a trained pathogenic learning classifier comprises applying an algorithm to the image data and expressed element data, wherein the algorithm predicts whether the hair sample comprises a combination of elements, thereby predicting one of a healthy and unhealthy condition and uses a covariate analysis to adjust for cellular content variation in said hair sample.

6. The method of claim 5, wherein said covariate analysis adjusts for a signal strength of one or more cell types.

7. The method of claim 6, wherein said one or more cell types comprises follicular cells, and abnormal pathogenic hair structure relating to fungal and pathogenic infection.

8. The method of claim 7, further comprising identifying a presence of said one or more cell types, cell deformities and cell dysplasia in said sample.

9. The method of claim 8, wherein said presence of said one or more cell types in the hair sample is identified by a presence of one or more biomarkers.

10. The method of claim 9, wherein said trained pathologist is trained with a training set comprising a training sample having healthy and unhealthy hair images, with a pathology selected from the group consisting of: subclinical fungal infection, preclinical fungal infection, pathogenic diseases of hair, and surrounding hair tissue.

11. The method of claim 1, wherein the unhealthy condition indicates a fungal infection caused by Aspergillus niger and a melanotic fungi.

12. The method of claim 1, wherein the unhealthy condition indicates a co-infection of Borrelia burgdorferi and a fungal organism.

13. The method of claim 12, wherein co-infection is identified by a symbiotic relationship between said Borrelia burgdorferi and said fungal organisms.

14. The method of claim 1, wherein the method employs the use of photon electromagnetic spectrum with light in multiple directions and wavelengths and angles to visualize the hair sample with clarity and without the use of stains.
